(19) United States
(12) Patent Application Publication
(10) Pub. No.: US 2010/0119648 A1
(43) Pub. Date: May 13, 2010

(54) LACTOSE-POSITIVE RECOMBINANT LEUCONOSTOC STRAIN

(75) Inventors: Peter Ruhdal Jensen, Gentofte (DK); Soren Helmark, Kgs. Lyngby (DK)

Correspondence Address: DARBY & DARBY P.C., P.O. BOX 770, Church Street Station, New York, NY, 10008-0770, US

(73) Assignee: DTU, TECHNICAL UNIVERSITY OF DENMARK, Lyngby (DK)

(21) Appl. No.: 11/908,478

(22) PCT Filed: Mar. 15, 2006
(86) PCT No.: PCT/DK2006/000150

§ 371 Date: Dec. 11, 2007

Related U.S. Application Data
(60) Provisional application No. 60/662,111, filed on Mar. 15, 2005.

(30) Foreign Application Priority Data
Mar. 15, 2005 (DK) PA 2005 00372

(51) Int. Cl. A23C 9/127; C12N 1/21; A23C 19/032; A23C 17/02

(52) U.S. Cl. 426/36; 435/252.3; 426/43; 426/41; 426/580; 426/582; 426/583

(57) ABSTRACT

The present invention provides a Leuconostoc bacterial strain for use in the diary industry to prevent the growth of pathogenic micro-organisms and thereby ensure the quality and safety of milk products produced by fermentation, including cheese, yoghurt, sour cream, buttermilk and kefir products. The Leuconostoc bacterial strain of the invention is a recombinant Leuconostoc carnosum strain, characterised by a lactose-positive phenotype with the ability to utilise lactose as sole carbon source. The invention provides a starter culture comprising the lactose-positive Leuconostoc carnosum of the invention for use in the manufacture of milk products, and milk products prepared with said Leuconostoc strain or said starter culture.

### BACKGROUND OF THE INVENTION

Improved methods and tools for protecting food products against spoilage by bacteria, moulds and other undesirable organisms and that can minimize or replace the use of chemical preservatives are of great importance to the food industry. Among the most serious causes of food spoilage is Listeria, a gram positive facultative anaerobe that is widespread in the environment. In particular the species Listeria monocytogenes is now recognized to be one of the most important food-borne pathogens and is often isolated from raw meat, vegetables, raw milk, and milk products, particularly cheese which may also be contaminated during processing. L. monocytogenes is known to be a human pathogen causing the potentially fatal disease listeriosis, while other species such as L. inanovii may also be human pathogens. L. monocytogenes is acid-, salt- and psychro-tolerant, and thus the proliferation of L. monocytogenes in food cannot be prevented by refrigerated storage or the addition of acid or salt.

Lactic acid bacteria are widely used in the food industry due to their ability to preserve food and add desirable flavour and texture to products. Leuconostoc species are lactic acid bacteria which have particular commercial importance in the dairy and meat industries. One attribute of some Leuconostoc species of value to industry is the ability to produce bacteriocins, which are peptides that exhibit antagonistic activities against a range of closely related bacteria. The growth of pathogenic micro-organisms in food can be prevented by the addition of bacteria that produce bacteriocins with antagonistic activity against the pathogenic micro-organisms.

The bacteriocin producing bacterium Leuconostoc carnosum is used in industry for bio-preservation of meat products because of its ability to prevent the growth of the food borne pathogen L. monocytogenes, while not imparting any undesirable flavour or odour to the product. When Leu. carnosum is added to sliced meat products (WO02/056694), it effectively prevents the growth of L. monocytogenes as its bacteriocins are active against all tested serotypes of L. monocytogenes (Budde, B. B., et al. (2003). International Journal of Food Microbiology. 83: 171-184; Keppler, K., et al., (1994) Food Microbiology. 11: 39-45; van Laack, R. L. J. M., et al., (1992) International Journal of Food Microbiology. 16: 183-195).

L. monocytogenes also thrives in dairy products, in particular in soft cheeses. Contamination by L. monocytogenes may originate from the raw milk, particularly from un-pasteurised milk products, but may also be introduced post-pasteurisation, during the many handling steps involved in milk processing, e.g. cheese making. L. monocytogenes is able to adhere to many different types of surfaces by forming a viable biofilm, and once a diary plant is contaminated, removal of such biofilms has proven to be highly resistant to standard sanitation methods. There is thus a recognised need in the dairy industry to find safe and effective means to control undesirable and/or pathogenic micro-organisms in milk products, and an attempt to screen for suitable bacteriocin-producing lactic acid bacteria has been reported (Buyong et al., 1998 Applied and environmental Microbiology 64: 4842-4845). In the absence of finding bacteriocin-producing lactic acid bacteria capable of growth on milk, there have been attempts to employ a bacteriocin-sorbic acid containing composition, or a sorbic acid-bacteriosin-producing micro-organism composition as a food additive (U.S. Pat. No. 6,780,447). This approach has the drawback that bacteriocins have a short half-life, being susceptible to proteolytic degradation. Thus there is a particular need to develop a commercial starter culture for the diary industry, which can ensure the quality and safety of milk products including yoghurt and cheese products.

### SUMMARY OF THE INVENTION

The present invention addresses the need for an anti-Listerial micro-organism that can be used in the diary industry to secure the safety and shelf-life of milk products. Leu. carnosum, that is currently used for the preservation of meat products, cannot be employed to prevent the growth of L. monocytogenes in milk products due to its inability to grow in milk. This inability to grow on milk is due to the fact that Leu. carnosum can not ferment lactose, which is the main carbon- and energy-source in milk. However, a lactose-positive strain of Leu. carnosum, that is able to grow in milk and milk products, would provide a novel and valuable means for preventing L. monocytogenes contamination and growth in dairy products.

Thus, the present invention provides an isolated recombinant Leuconostoc carnosum strain capable of growth on milk, or a milk product, that is useful for the natural preservation of dairy products. The isolated recombinant Leu. carnosum strain of the invention is characterised by its ability to grow on lactose as sole carbon source. The recombinant Leuconostoc carnosum strain is further characterised by its ability to produce a bacteriosin, which is toxic to pathogenic micro-organisms found in dairy products, in particular Listeria monocytogenes.

In one embodiment the recombinant Leuconostoc carnosum strain of the invention comprises one or more genes encoding a lactose transporter and/or a β-galactosidase or β-glucosidase. Accordingly, the invention encompasses a Leuconostoc carnosum strain that is a mutant, produced by mutagenesis of one or more native gene of the Leuconostoc carnosum strain.

In an alternative embodiment, the genes in the recombinant Leuconostoc carnosum strain, that encode a lactose transporter and/or β-galactosidase or β-glucosidase, are heterologous genes that are stably inherited in the Leuconostoc carnosum strain.

The invention provides a recombinant Leuconostoc carnosum strain comprising a gene encoding a β-galactosidase polypeptide having an amino acid sequence at least 60% homologous to SEQ ID NO: 3. Preferably said β-galactosidase is encoded by a nucleic acid molecule that hybridises to a nucleic acid molecule with a nucleotide sequence consisting of SEQ ID NO: 1 or 4, under stringency conditions of no less than 1×SSC at 65° C. The invention further provides a recombinant Leuconostoc carnosum strain comprising a gene encoding a lactose transporter polypeptide having the amino acid sequence at least 60% homologous of SEQ ID NO: 2, or a fragment thereof, having functional lactose transporter activity. Preferably said lactose transporter is encoded by a nucleic acid molecule that hybridises to a nucleic acid molecule with a nucleotide sequence consisting of SEQ ID NO: 1 or 6, under stringency conditions of no less than 1×SSC at 65° C. In an alternative embodiment, the recombinant Leuconostoc carnosum strain of the invention comprises a nucleic acid molecule having the nucleotide sequence of SEQ ID NO: 1, that comprises a coding sequence for a lactose transporter and a β-galactosidase.

In one embodiment, the recombinant Leuconostoc carnosum strain comprises a nucleic acid molecule encoding a lactose transporter and a β-galactosidase that is harboured on a self-replicating plasmid, and in an alternative embodiment the nucleic acid molecule is integrated into the genome (including a bacterial chromosome and/or a native plasmid) of said strain. The nucleic acid molecule, encoding a lactose transporter and a β-galactosidase, may be comprised within an operon, and furthermore be operably linked to a promoter.

The present invention further provides a chemically defined growth-medium, (Table 3, supplemented with a carbon source e.g. lactose) for the growth and selection of Leuconostoc carnosum, including the selection of a lactose-positive Leuconostoc carnosum strain.

The invention further provides a starter culture for use in the production of milk products, comprising a recombinant Leuconostoc carnosum strain of the invention that is characterised by its ability to grow on lactose as sole carbon source; and encompasses the use of said starter culture or recombinant Leuconostoc carnosum strain in the production of a milk product.

In a further embodiment, the invention encompasses a milk product comprising a recombinant Leuconostoc carnosum strain of the invention, characterised by its ability to grow on lactose as sole carbon source, wherein the milk product is selected from cheese, butter milk, sour cream, yoghurt and kefir.

Furthermore a method is provided for constructing a recombinant Leuconostoc carnosum strain of the invention comprising the steps of: transforming Leuconostoc carnosum with at least one nucleic acid molecule encoding a lactose transporter polypeptide and a β-galactosidase polypeptide, and selecting transformed cells of Leuconostoc carnosum characterised by the ability to grow on lactose as sole carbon source.

### BRIEF DESCRIPTION OF THE FIGURES

FIG. 1: Growth of Leu. carnosum 4010 in the chemically defined medium (Table 3) supplemented with either 1% (w/v) glucose (minimal medium: −) or 1% (w/v) glucose and 0.34 g YNB per liter (minimal medium+YNB: ×). Growth followed by OD 620 measurements over time (minutes).

FIG. 2. Growth in chemically defined medium (Table 3) of recombinant Leu. carnosum SH0020 ( [symbol] ) with 1% (w/v) lactose compared to Leu. carnosum 4010 ( [symbol] ) with 1% (w/v) glucose. Growth followed by OD 600 measurements over time (minutes).

FIG. 3. Specific growth rates of lactose positive recombinant strains of Leu. carnosum in chemically defined medium (Table 3) supplemented with 1% (w/v) lactose.

### DEFINITION/ABBREVIATION OF TERMS

### Growth Media:

ABT agar: containing 1 mM MgCl₂, 0.1 mM CaCl₂, 0.01 mM FeCl₃, 15 mM (NH₄)₂SO4, 41.68 mM Na₂HPO₄, 22 mM KH₂PO₄, 51.33 mM NaCl, 2.5 mg/l Thiamin and 20 g/l Bacto Agar, Difco Laboratories, Detroit, USA.

BHI broth: Brain heart infusion supplied by Oxoid Ltd, Basingstoke, Hampshire, England.

GM17 agar: comprising 1% (w/v) glucose and 48.25 g/l M-17 agar supplied by Oxoid Ltd, Basingstoke, Hampshire, England.

LB: Luria-Bertani medium containing 4 g/l NaCl, 5 g/l Yeast extract supplied by Difco, Detroit, USA, 10 g/l Peptone from pancreatically-digested casein supplied by Merck KGaA, Darmstadt, Germany and 20 g/l Bacto Agar, Difco Laboratories, Detroit, USA.

SA agar: Synthetic Amino acid agar. The composition of this medium is given in Jensen, P. R. and Hammer, K. (1993) Applied and Environmental Microbiology. 59: 4363-4366.

YNB: Yeast Nitrogen Base w/o amino acids and ammonium sulphate, Difco Laboratories, Detroit, USA.

### Additional Terms:

Bacteriocin: a peptide produced by Leuconostoc species that is toxic to Listeria monocytogenes.

CAM: chloramphenicol

Hybridisation stringency: a nucleic acid molecule will hybridise with another nucleic acid molecule sharing sequence homology to form a hybrid, as for example during a Southern Hybridisation assay. The stability of the formed hybrid, under conditions of increasing stringency during the washing step of the assay, is a measure of the sequence homology between the hybridising molecules. Washing under high stringency conditions corresponds to at least 1×SSC at 65° C. Optionally, the washing step can be performed at 0.5×SSC at 65° C.

Leuconostoc strains: include Leuconostoc carnosum, also designated as Leu. carnosum; and Leuconostoc lactis also designated as Leu. lactis.

PCR: polymerase chain reaction performed according to the following general procedure: Two reaction mixes are prepared on ice:

Mix 1: 5 μl 5 mM dNTP-mix, 3 μl 10 μl Forward primer, 3 μl 10 μl Reverse primer, 3 μl 5 ηg/ml template DNA and 31 μg H₂O.

Mix 2: 5 μl Elongase enzyme mix provided by Invitrogen, 25 μl Buffer B provided by Invitrogen and 45 μl H₂O.

20 μl of mix 1 and 30 μl of mix 2 are mixed together and the following amplification is performed using a PCR thermocycler:

| Step no. | time (min) | Temperature (° C.) |
|---|---|---|
| 1 | 4 | 94 |
| 2 | 1 | 94 |
| 3 | 1 | 50 |
| 4 | 5 | 68 |

Steps number 2-4 are repeated 30 times, whereafter another 20 μl of mix 1 and 35 μl of mix 2 (without the template DNA) are added to the reaction mix and one more cycle of steps 1-4 is performed.

PTS: phosphoenolpyruvate-dependent phosphotransferase is a system which facilitates the transport of mono- and di-saccharides.

Recombinant: comprises genetic variation in a cell (including bacterial) resulting from a recombinant event such as mutation (random genetic change within a cell's own genetic code). Bacterial mechanisms for exchanging genetic material may also result in a recombinant bacterium that contains a combination of traits from two different parental cells. Different modes of exchange identified in bacteria include:
1. transformation (the transfer of naked DNA from one bacterial cell to another in solution, including dead bacteria),
2. transduction (the transfer of viral, bacterial, or both bacterial and viral DNA from one cell to another via bacteriophage) and;
3. conjugation (the transfer of DNA from one bacterial cell to another via a conjugation pilus).

Recombinant bacteria may also result from the transfer and stable inheritance of foreign DNA produced by genetic engineering. Bacteria, having acquired DNA from any of these events, can then undergo fission and pass the recombined genome to new progeny cells.

Starter culture: a culture of one or more food grade lactic acid bacterial strains suitable for addition to milk or milk products in the manufacture of diary products and beverages (e.g. kefir, drinking yogurt).

Vector: nucleic acid molecule employed for cloning and optionally expression of a gene. The vector is either capable of self-replication, e.g. plasmid, or is non-self-replicating in a host cell (e.g. Leu. carnosum) and functions as an integration plasmid.

X-gal: 5-bromo-4-chloro-3-indolyl-β-D-galactopyranoside is a non-inducing chromogenic substrate for β-galactosidase, which hydrolyzes X-Gal forming an intense blue precipitate. X-Gal is utilized for detection and/or selection of β-galactosidase gene activity in transfected/transformed cells.

### DETAILED DESCRIPTION OF THE INVENTION

### Metabolic Properties of Leuconostoc:

Leuconostoc is a facultative anaerobe, depending on mono- or di-saccharides as a fermentable source of energy. All Leuconostoc strains ferment glucose, although the majority of strains prefer fructose. Leuconostoc belongs to a subgroup of lactic acid bacteria where the end product of fermentation is less that 90% lactic acid, and may include large amounts of ethanol, acetate and CO₂, produced by a form of heterofermentation (Garvie, E I., (1986) Bergey's Manual of Systematic Bacteriology, vol. 2, 9^{th} ed. Eds: Sneath, PHA et al., Williams & Wilkins, Baltimore, p. 1071-1075). Heterofermentative organisms have no perfect glycolysis due to the absence of the glycolytic enzyme fructose 1,6-diphosphate aldolase, instead relying on a combination of glycolysis and the phosphoketolase pathway to break down glucose. The nutritional requirements of lactic acid bacteria, including some Leuconostoc strains, has been shown to be complex, with a requirement for vitamins, amino acids, purines and pyrimidines, which may account for their limited natural distribution.

### Growth on Lactose as Sole Carbon Source:

Leuconostoc carnosum subspecies have been shown to ferment the sugars cellobiose, fructose, glucose, mannose, mellibiose, ribose, salicin, sucrose and trehalose, but not lactose. The ability to utilise a fermentable carbohydrate source relies on a transport mechanism for intracellular import of the carbohydrate energy source or its degradation product, and a metabolic pathway for its catabolism. At least three mechanisms for the transport of sugars are known to occur in prokaryotes, namely a permease system, an ATP-binding cassette transporter (ABC transporter), and a phosphoenol pyruvate-dependent phosphotransferase system (PTS).

The PTS system for lactose import in Lactococcus lactis and Lactobacillus casei is characterised by a trans-membrane protein EIIBC that phosphorylates lactose and transports it into the cell. A phosphorylation chain, comprising Enzyme 1, coupled to histidine protein, coupled to Enzyme IIA, serves to transfer a phosphoryl group from phosphoenol pyruvate through to the transmembrane protein EIIBC (de Vos, W M and Vaughan, E E (1994) FEMS Microbiology Reviews 15: 217-237). The pathway is energetically favourable because PTS mediated lactose uptake has no net ATP cost. The genes encoding this PTS pathway are known, and in L. lactis they are located on a so-called ‘lactose plasmid’ which carries the lac operon for utilization of lactose and galactose.

The ATP transporter comprises two transmembrane domains, which mediate sugar import, designated as LacF and LacG in Agrobacterium radiobacter, and two cytoplasmic domains (LacK/LacK) that provide energy required to drive uptake via ATP hydrolysis (de Vos, W M and Vaughan, E E (1994) supra).

The permease system transports protons or Na⁺ ions and mono- or di-saccharides across the cell membrane. Transport is driven by proton motive force that is generated by a proton or Na⁺ gradient maintained by a H⁺ ATPase pump (de Vos, W M and Vaughan, E E (1994) supra). The permease system is known to operate as a proton-sugar symporter or as a sugar-sugar anti-porter. The LacS permease system of Streptococcus termophilus is known to operate in two modes, either as an antiporter, whereby LacS transports lactose into the cell and exports galactose, or a galactose/H⁺ symporter. Lactose, imported into the cell, is cleaved by β-galactosidase into glucose and galactose, where galactose export is seen to occur in cells that are unable to metabolise galactose. In lactic acid bacteria, lactose imported into the cell may be further hydrolysed by β-galactosidase into glucose and galactose, where glucose enters the glycolytic and phosphoketolytic pathway, and galactose is metabolised by the Leloir pathway. Accordingly uptake and fermentation of lactose, imported via the permease system, may involve both a permease and a β-galactosidase, coupled to downstream metabolic pathways. Although Leu. carnosum is lactose-negative, other Leuconostoc species can transport lactose via the permease pathway, e.g. Leu. lactis that has a lacS encoded permease, and a lacLM encoded β-galactosidase. Both the lacS and lacLM genes have been found on a plasmid (pNZ63) isolated from the Leu. lactis strain NZ6009/DMS 20202. The Leloir pathway enzymes for galactose metabolism in Leu. lactis are encoded by chromosomal genes. In contrast, in S. termophilus, the genes encoding both LacS, β-galactosidase and the Leloir pathway enzymes, are known to be harboured on a plasmid.

Isolated Leu. carnosum Strain Able to Grow on Lactose as Sole Carbon Source:

The present invention provides a lactose-positive recombinant strain of Leu. carnosum which, in one embodiment, has been constructed by introducing genes, coding for both lactose uptake and utilization, into Leu. carnosum by transformation. Gene encoded functions required to confer a lactose-positive phenotype in Leu. carnosum include at least a lactose transporter, and preferably also an enzyme that catalyses the hydrolysis of lactose or lactose-phosphate, for example, but not limited to, a β-galactosidase or β-glucosidase. Genes encoding lactose transporters that may be introduced into Leu. carnosum include both PTS, ABC transporter and permease genes.

Preferred examples include the PTS encoding genes of the lactose operon of L. lactis, which is harboured on the ‘lactose plasmid’. For example, the plasmid pool from L. lactis FHCY-1, comprising the ‘lactose plasmid’, is isolated and each plasmid in the pool is fused with the replicon and the selective antibiotic marker from the vector pCl372, and transformed into Leu. carnosum. Lactose positive recombinant strains of Leu. carnosum are selected by screening for chloramphenicol resistance and the ability to grow on lactose as the sole sugar source.

Preferred examples of permease encoding genes that may be used to provide a lactose-positive recombinant Leu. carnosum strain, include lacS from Leuconostoc lactis, Lactobacillus plantarum or Streptococcus termophilus. Genes encoding β-galactosidase, which may be introduced into Leu. carnosum, include lacLM from Leuconostoc lactis, the lacA from Lactobacillus plantarum, LacG from Streptococcus termophilus or LacZ from Streptococcus termophilus, Lactococcus lactis or E.coli.

The genes encoding LacS (a transporter for lactose) and LacLM (a β-galactosidase) that may be employed in the present invention are both present on the plasmid pNZ63 from Leu. lactis DMS 20202. Likewise a ‘lactose plasmid’ with genes for the utilization of lactose is present in Leu. lactis CHCC 1990 and can thus be used for transformation into Leu. carnosum to engineer a lactose positive strain.

One or more genes, of known origin, encoding functions required to confer a lactose-positive phenotype may be introduced into Leu carnosum by means of transformation. The nucleic acid molecule comprising said genes may be obtained from genetic material (DNA/RNA) isolated from its source of origin, employing a variety of standard molecular biology procedures, known to a skilled person. In brief DNA (or cDNA derived from RNA), isolated from its source of origin, is restriction digested and cloned as a DNA library in a suitable vector and amplified in a host cell. A clone comprising the gene of interest may subsequently be screened out from the DNA library using standard DNA screening protocols (including colony/DNA hybridisation), thereby providing a pure source of the required nucleic acid molecule. Alternatively, the nucleic acid molecule comprising the gene of interest may be amplified from said genetic material employing for example PCR based technology, using primers homologous to the 5′ and 3′ termini sequence of the respective gene. In a further approach, a synthetic gene may be assembled from synthetic nucleic acid molecules, having a nucleotide sequence corresponding to the gene of interest.

Nucleic acid molecules comprising one or more genes encoding functions required to confer a lactose-positive phenotype, comprise at least one coding sequence domain, encoding at least one function, that is functionally linked to a promoter domain, wherein said two domains may be of homologous or heterologous origin with respect to each other. The genes encoding functions that confer a lactose-positive phenotype may be located in an operon that is operably-linked to a promoter domain. A promoter domain capable of directing expression of said coding sequence domain is functionally linked to said coding sequence. For example, said coding sequence domain may be functionally linked to a library of synthetic promoter sequences, from which one or more promoter domain functionally linked to said coding sequence domain, and having the desired level of promoter strength (transcription activity) can be selected, as illustrated in Example 1 and described by Solem C and Jensen P R (2002) Applied and Environmental Microbiology 68: 2397-2403.

Nucleic acid molecules may be transformed into Leu. carnosum by means of transformation, either directly, or following cloning into a suitable plasmid. Suitable plasmids that replicate and are stably maintained in Leu. carnosum include pG+host8 (TET^{R}) (Maguin, E., Prévost, H., Ehrlich, S. D. and Gruss, A. (1996) Journal of Bacteriology 178: 931-935); pCI3340 (CAM^{R}), and pCI372, (Hayes, F., Daly, C. and Fitzgerald, G. F. (1990) Appl. Environ. Microbiol. 56: 202-209) and are thus suitable plasmids for harbouring genes required to confer a lactose-positive phenotype. It may furthermore be desirable to employ a plasmid which does not include any antibiotic resistance genes, e.g. by selecting for transformants with a lac+ phenotype conferred by the plasmid carrying the lactose genes, as shown in example 3. The given examples serve to illustrate the steps of isolating and/amplifying nucleic acid molecules comprising genes encoding lactose transport and β-galactosidase functions, their insertional cloning into a vector, and the transformation of the recombinant vector into a Leu. carnosum 4010 cell (Budde et al., (2003) International Journal of Food Microbiology. 83: 171-184). Said nucleic acid molecules may be maintained in a host Leu. carnosum cell by virtue of being harboured on a stable, maintained plasmid. Alternatively, genes encoding lactose transport and β-galactose functions may be stably integrated into the genome of Leu. carnosum. Briefly, nucleic acid molecules comprising said genes are cloned into a vector that is not capable of self-replication in Leu. carnosum. The recombinant vector is transformed into Leu. carnosum, followed by selection of lac+, gal+ recombinant cells. Integration events are facilitated by homologous recombination between homologous sequences present in both the Leu. carnosum genome and in vector sequences flanking said genes. Transformation protocols suitable for the introduction of a vector or nucleic acid molecule into Leu. carnosum are described in Example 2 and Helmark S et al., (2004) Appl. Environmental Microbiol. 70: 3695-3699. In a further embodiment, natural plasmids containing the lactose genes found in bacteria closely related to Leu. carnosum, e.g. Leuconostoc lactis and Lactococcus lactis, may be transferred to Leu. carnosum by conjugation or by natural transformation.

As illustrated in the given examples, a recombinant Leu. carnosum strain with a lactose positive phenotype, may be obtained by introducing at least one nucleic acid molecule encoding a functional lactose transporter, and preferably also a β-galactosidase or β-glucosidase, into a cell of Leu. carnosum. A recombinant Leu. carnosum strain of the invention thus includes a cell that comprises a nucleic acid molecule encoding a lactose transporter polypeptide having an amino acid sequence that is at least 60%, 65%, 70%, 75%, 80%, 85%, 90% or 95% homologous to the amino acid sequence of SEQ ID NO: 2, or a fragment thereof, conferring functional lactose transporter activity. A nucleic acid molecule encoding said lactose transporter encompasses a nucleic acid molecule that encodes a lactose transporter conferring functional lactose transporter activity and having a nucleic acid sequence that is at least 60%, 65%, 70%, 75%, 80%, 85%, 90% or 95% homologous to the nucleic acid sequence of SEQ ID NO: 6.

Furthermore, a recombinant Leu. carnosum strain of the invention includes a cell that comprises a nucleic acid molecule encoding a β-galactosidase polypeptide having an amino acid sequence that is at least 60%, 65%, 70%, 75%, 80%, 85%, 90% or 95% homologous to the amino acid sequence of SEQ ID NO: 3, or a fragment thereof, conferring functional β-galactosidase activity. A nucleic acid molecule encoding said β-galactosidase encompasses a nucleic acid molecule that encodes a β-galactosidase conferring functional β-galactosidase activity and having a nucleic acid sequence that is at least 60%, 65%, 70%, 75%, 80%, 85%, 90% or 95% homologous to the nucleic acid sequence of SEQ ID NO: 4.

In an alternative embodiment, a recombinant Leu. carnosum strain expressing a lactose positive phenotype is obtained by mutagenic activation of any native cryptic genes encoding proteins required for lactose metabolism and transport present in the Leu. carnosum genome. Suitable mutagenic treatments, well known in the art include, for example, treatment with a chemical mutagen such as ethanemethane sulphonate (EMS) or N-methyl-N′-nitro-N-nitroguanidine (NTG), or exposure to UV light.

Selection of recombinant Leu. carnosum cells that are mutants, or are transformed with genes that confer a lactose positive phenotype and β-galactosidase activity may be selected on appropriate growth media: namely a minimal medium or a chemically defined growth-medium supplemented with lactose as sole fermentable sugar carbon in order to select for lactose transport and fermentation; and a growth-medium supplemented with x-gal to detect β-galactosidase activity. The present invention provides the first described chemically defined growth-medium for Leu. carnosum, described in Example 3, which may be supplemented with lactose as sole fermentable sugar and used for selecting and maintaining lactose-positive recombinant Leu. carnosum cells.

Lactose-positive recombinant Leu. carnosum cells may be detected and identified by virtue of their lactose-positive and x-gal positive phenotype. In addition they may be identified by virtue of the presence of heterologous genes encoding lactose permease and β-galactosidase functions. Thus, molecular probes, capable of hybridising to the heterologous genes encoding these functions may be used to identify cells having genetic material comprising these genes, employing standard detection techniques, including DNA hybridisation and PCR amplification and transcript profiling.

### Leuconostoc Species Produce Toxic Bacteriocin Peptides:

The invention is directed to lactose-positive recombinant Leu. carnosum and its use in the bio-preservation of milk and milk products. The Leuconostoc species, Leu. carnosum, has been found to produce bacteriocins that are particularly toxic to Listeria monocytogenes. Bacteriocins form pores in the membranes of a sensitive target cell, thereby depleting the trans-membrane potential and pH gradient across the cell membrane and causing leakage of intracellular material. The bacteriocins produced by Leu. carnosum belong to Class IIa bacteriocin peptides (also known as carnocin or leucocin), characterised by having a molecular mass of <5 kDa, heat-stability, and the consensus sequence YGNGVXaaC in the N-terminal part of the peptide, associated with a disulfide bridge. Thus the lactose-positive recombinant Leu. carnosum cell of the invention is a cell capable of producing a bacteriosin, and preferably a bacteriosin belonging to the Class IIa bacteriosin. In a particularly preferred form of the invention the lactose-positive Leu. carnosum cell produces two bacteriosins classified as leucocin A-4010 and leucocon B-4010 (Budde et al., (2003) International Journal of Food Microbiology. 83: 171-184). The N-terminal sequences of these peptides are KYYGNGVHCTKSGCSVNWGEAFSAGVHRLA—(leucocin A-4010) and KNYGNGVHCTKKGCSVDWGYAWTNIANNSVMNGLTGGNA—(leucocin B-4010) (Budde et al., (2003) International Journal of Food Microbiology. 83: 171-184). The disclosed invention may be practised with any recombinant Leu. carnosum cell having a lactose-positive phenotype that may be constructed or produced according to the described methods. Sub-species of Leu. carnosum that may provide a suitable host cell for the construction of a lactose-positive strain include Leu. carnosum 4010 (CHCC 1043) obtainable from Chr.Hansen A/S, 10-12 Boge Allé, DK 2970 Horsholm as B-SF-43, sold under the trade name Bactoferm; LA44A, LA54A, Tal1a.

Starter Cultures Comprising Lactose-Positive Recombinant Leu. carnosum

The lactose-positive recombinant Leu. carnosum cell of the invention may be used as a food-grade starter culture in the manufacture of fermented milk products. A starter culture, comprising the lactose-positive recombinant Leu. carnosum strain of the invention, may be a pure culture of said strain or alternatively may include a mixture of one or more additional strains of different lactic acid bacterial species, such as Streptococcus and Lactobacillus species. The selection of strains to be combined with the Leu. carnosum strain of the invention will depend on the type of dairy product to be manufactured. Mesophilic cultures comprising species of Lactococcus, Leuconostoc, and Lactobacillus are preferred for cheese and butter manufacture, whereas cultures of thermophilic species of Streptococcus and Lactobacillus are preferred for fermented milk products such as yoghurt. Starter cultures of the present invention may also include fungal cultures, for use in the manufacture of certain types of cheese, for example Penicillum roqueforti, Penicillum candidum and Geotrichum candidum.

Starter cultures according to the invention may be prepared as concentrates which may be stored and distributed in frozen form, without loss of viability. Alternatively the starter culture may be freeze-dried or lyophilised, or alternatively stored in liquid form with a preservative. Starter cultures, according to the invention, whether liquid or dried, may be added directly into milk or dairy products as direct vat set (DVS) cultures. However, it is not uncommon in the dairy industry to prepare and maintain bulk starter cultures. Thus the starter culture according to the present invention may be inoculated into heat-treated milk and incubated to allow propagation of the starter culture strains to the desired cell number, in order to prepare a ‘bulk starter’. Alternatively, the starter culture of the invention is pre-activated, to diminish its lag-phase of growth, by incubation in a small volume of pre-heated milk for 30 to 60 minutes before adding to the milk for manufacture of a dairy product.

A food-grade starter culture according to the invention, comprising lactose-positive recombinant Leu. carnosum, may be used in the manufacture of a fermented milk product including: cheese (pasteurised and un-pasteurised cheese); soft or hard cheese; ripened cheese (including Cheddar, Colby, Edam, Muenster, Gruyere, Emmanthal, Camembert, Parmesan and Romano); blue cheese (including Danish blue and Roquefort); fresh cheese (including mozzarella and Feta); acid coagulated cheese (including cream cheese, Neufchatel, Quarg, Cottage cheese and Queso Blanco); and pasta filata cheese; and other cheese types (Campesino, Chester Danbo, Drabant, Herreg\dot{a}rd, Manchego, Provolone, Saint Paulin); buttermilk; sour cream; yoghurt; and kefir.

The starter culture of the invention can be employed in a cheese making process that may comprise the steps of:
equilibrating milk (optionally pasteurised milk) to a temperature suitable for the selected starter culture,
addition of the starter culture to the milk and incubation to allow fermentation to form a cheese milk with the desired fall in pH,
coagulation of the cheese milk to produce milk curds by either addition of rennet, or acid (and optionally heat) treatment,
cutting, draining, pressing of the milk curds, with optional addition of salt.

### Example 1

### Construction of Lactose Positive Recombinant Strains of Leu. Carnosum Comprising Streptococcus thermophilus DNA Sequences Encoding a Lactose Uptake Transporter and β-galactosidase

For the production of lactose-positive recombinant strains of Leu. carnosum, the following strategy was applied: A plasmid (pSH101) harbouring the genes for utilization of lactose was constructed in the vector pCI372 according to a cloning strategy detailed below, and pSH101 was then introduced in Leu. carnosum by means of electroporation and natural transformation. The vector pCI372, that is described by Hayes et al, (1990) supra and Lucey M, et al, (1993) FEMS Microbiol Lett. 110(3): 249-56, was chosen because it has been shown to replicate in Leu. carnosum (Helmark, S., et al., (2004) Applied and Environmental Microbiology. 70: 3695-3699.

Protocol for cloning genes for lactose utilisation: The lacSZ nucleic acid molecule [SEQ ID NO:1] contains two genes encoding a lactose uptake transporter [SEQ ID NO: 2], and a β-galactosidase [SEQ ID NO:3]. The two genes present in the lacSZ nucleic acid molecule are individually the lacZ gene [SEQ ID NO: 4], encoding a β-galactosidase [SEQ ID NO:5], and the lacS gene [SEQ ID NO: 6], encoding a lactose uptake transporter [SEQ ID NO: 7]. The lacSZ nucleic acid molecule [SEQ ID NO:1] was obtained by means of PCR amplification of chromosomal DNA purified from Streptococcus thermophilus (obtained from an isolate from a “sodmaelksyoghurt” from Arla Foods amba, Viby J., Denmark. PCR was performed with a forward primer mixture comprising a synthetic randomized promotor sequence (Solem C and Jensen P R, 2002 supra):

5′ACGACTAGTGGATCCATNNNNNAGTTTATTCTTGACANNNNNNNNNNN
NNNTGRTATAATNNNNAAGTAATAAAATATTCGGAGGAATTTTGAAATGG
AAAAATCTAAAGGTCAG -3′
(wherein N = 25% each of A, C, G and T; R = 50%
each of A and G; SEQ ID NO: 8)
and
a reverse primer:

(SEQ ID NO: 9)

5′-GGTACTCGAGGAAGATACTAACACACTAATG-3′,

thereby yielding a mixture of lacSZ gene fragments fused to promotors of varying strengths.

The fragment mixture was digested with BamHI and XhoI and the vector pCI372 was digested with BamHI and SalI (Sal1 is compatible with Xho1).

The vector DNA was further treated with Shrimp Alkaline Phosphatase (SAP) to prevent re-ligation of the cloning vector. Subsequently, the fragment mixture and the SAP-treated vector DNA were ligated overnight at 16° C. using T4 DNA ligase and standard ligation conditions. Similarly, the fragment mixture was ligated under the same conditions to vector DNA that had not been treated with SAP, as a control to monitor vector self-ligation.

Ligation mixtures were transformed into E. coli MC-1000 (Boogerd, F. C., et al. (1998) J. Bacteriol., 180, 5855-5859) and Lactococcus lactis spp. cremoris MG-1363 (Gasson M J. (1983) J Bacteriol. April;154(1):1-9) by electroporation and after a phenotypic induction in BHI broth supplemented with 0.25M sucrose, 27.8 mM glucose, 20 mM MgCl₂ and 2 mM CaCl₂ for a period of two hours, transformants were selected on either lactose minimal agar plates (ABT agar with 1% (wlv) lactose for E. coli and Synthetic Amino acids (SA) containing 1% (w/v) lactose for L. lactis) or agar plates containing chloramphenicol (CAM) and x-gal (LB containing 10 μg/ml CAM, 100 μg/ml x-gal and 1% (w/v) glycerol for E. coli and GM17 agar with the same supplements for L. lactis). The E. coli MC-1000 and L. lactis MG-1363 transformants obtained are shown in Table 1.

TABLE 1
Transformants of E. coli MC-1000 and L. lactis MG1363.

| | | | | CAM-x-gal plates | |
|---|---|---|---|---|---|
| | SAP-treatment | | Minimal | (total no. of | CAM-x-gal plates |
| Strain | of vector | Ligation | plates | colonies) | (blue colonies) |
| MC-1000 | Yes | 1Xligation | 0 | 5.E+04 | 2 |
| | No | 1Xligation | 9 | 5.E+04 | 0 |
| | Yes | Religation | 0 | 5.E+04 | 0 |
| | No | Religation | 6 | 5.E+04 | 0 |
| MG-1363 | Yes | 1Xligation | 80 | 1.E+04 | 21 |
| | No | 1Xligation | 92 | 1.E+04 | 15 |
| | Yes | Religation | 1 | 1.E+04 | 0 |
| | No | Religation | 0 | 5.E+03 | 0 |

(1Xligation = ligation mixture with a vector to fragment molar ratio of 1:1; religation = ligation mixture comprising vector DNA alone)

The SAP treatment of the vector prior to ligation is shown to be effective by the absence of E. coli MC-1000 colonies growing on minimal medium following transformation with SAP treated and self-ligated vector (re-ligation). Ligation of the lacSZ fragment into the pC1372 vector was confirmed by growth of L. lactis MG-1363 transformed with the ligated plasmid, but not the self-ligated vector, on lactose-supplemented minimal medium plates.

128 of the MG-1363 transformants were chosen for further study, comprising colony numbers: 1-47 selected on CAM-x-gal plates and colony numbers 48-128 selected on lactose minimal medium plates. Plasmid DNA was purified from the selected MG-1363 transformants in such a way that the purified DNA was pooled in four groups: 1) DNA from colony number 1 to 31; 2) from 32 to 62; 3) from 63 to 98; and 4) from 99 to 128 and each of the plasmid pools were transformed into Leu. carnosum using electroporation, as well as natural transformation (Helmark, S., et al., (2004) Applied and Environmental Microbiology. 70: 3695-3699) according to the following procedures:

Protocol for electroporation: Competent cells were obtained by growing a culture of Leu. carnosum 4010 overnight in BHI broth supplemented with 0.25 M sucrose, 27.8 mM glucose, and 1% (wt/vol) glycine. This culture was diluted in an appropriate volume of the same solution to give an initial optical density at 620 nm of preferably about 0.03 to 0.04 and then grown for between 1-2 generations, before transferring the culture to ice for 2 minutes and then harvesting the cells by centrifugation at 5,000×g for 5 minutes at 4° C. The harvested cells were washed in ice-cold washing solution comprising 0.25 M sucrose and 10% (wt/vol) glycerol, and then re-suspended in washing solution at a cell density corresponding to an OD 620 nm of 30, to provide competent cells for electroporation. A transformation mixture, comprising 50 μl sample of the Leu. carnosum 4010 competent cells and about 0.05 μg plasmid DNA, was transferred to a sterile electroporation cuvette with an inter-electrode distance of 0.2 cm, and stored on ice for 5 minutes. The transformation mixture in the cuvette was then subjected to a pulse of 25 μF, 200Ω, and between about 2.5 to 5.0 kV/cm, and immediately there after re-suspended to a final volume of 1 ml of pre-temperature equilibrated (about 18-25° C.) BHI broth supplemented with 0.25M sucrose, 27.8 mM glucose, 20 mM MgCl₂, and 2 mM CaCl₂, and incubated at 25° C. for 2 h to allow phenotypic expression of plasmid genes. The transformation mixture was then centrifuged at 4,000×g for 2 minutes. The pelleted cells were re-suspended in 1 ml 0.9% (wt/vol) NaCl, and dilutions were plated and grown at 25° C. on lactose-minimal agar plates comprising pre-treated BHI medium [medium that is pre-grown with Leu. carnosum in order to remove fermentable carbohydrates; filter sterilized; and supplemented with 1% (w/v) lactose]; and on CAM-x-gal agar plates comprising BHI agar containing 5 μg/ml CAM, 100 μg/ml x-gal and 1% (w/v) glycerol.

Protocol for natural transformation: transformation was performed essentially according to the above procedure for electroporation, with the exception that the electroporation pulse was omitted. The Leu. carnosum 4010 transformants obtained by electroporation or natural transformation are shown in Table 2.

TABLE 2
Transformants of Leu. carnosum 4010.

| | Electroporation (E)/ | | CAM-x-gal | CAM-x-gal |
|---|---|---|---|---|
| Plasmid | Natural | Lactose- | (total no. of | (% blue |
| pool | Transformation (NT) | Minimal | colonies) | colonies) |
| 1 | E | 0 | 498 | 50 |
| 2 | E | 5 | 762 | 66 |
| 3 | E | 1 | 329 | 45 |
| 4 | E | 0 | 544 | 95 |
| none | E | 0 | 0 | — |
| 1 | NT | 0 | 837 | 50 |
| 2 | NT | 0 | 597 | 75 |
| 3 | NT | 0 | 486 | 50 |
| 4 | NT | 6 | 499 | 95 |
| none | NT | 0 | 0 | — |

The Leu. carnosum 4010 colonies detected on CAM-x-gal plates varied significantly in size, with a diameter of up to 1.0 mm, where the smallest colonies had the strongest colour intensity. The colonies detected on lactose minimal plates had a diameter of 0.3-0.5 mm and were slightly yellowish. A total of 45 Leu. carnosum 4010 colonies, comprising colonies SH0001-SH0012 (selected on lactose-minimal plates and SH0013-SH0045 (selected on CAM-x-gal medium) were re-streaked on CAM-x-gal plates, and all colonies grew to a diameter of 0.1-1.0 mm with varying, but slightly reduced, colour intensity. The phenotype of nine of the β-galactosidase positive transformants was investigated by growth in a chemically defined medium for Leu. carnosum (see Example 3).

### Example 2

### Construction of lactose-positive recombinant strains of Leu. Carnosum comprising Leuconostoc lactis DNA sequences encoding a lactose uptake transporter and β-galactosidase.

A lactose-positive Leu. carnosum strain is obtained by transferring the genes encoding lactose permease and beta-galactosidase from the close relative, Leuconostoc lactis. In Leuconostoc lactis the gene for lactose transport; lacS [SEQ ID NO: 10] encoding a lactose uptake transporter [SEQ ID NO: 11] and the genes for lactose degradation; lacLM [SEQ ID NO: 12] encoding a β-galactosidase composed of the subunits LacL [SEQ ID NO: 13] and LacM [SEQ ID NO: 15] are located on a plasmid (pNZ63) described by Vaughan et al. (1996) Applied and Environmental Microbiology 62:1574-1582 and David et al. (1992) J. Bacteriology 174: 4475-4481. Three different approaches are used for transferring the Leuconostoc lactis lacS and lacLM genes into Leu. Carnosum as given below:

1) Conjugation: the Leuconostoc lactis lactose plasmid (pNZ63) is transferred to Leu. carnosum by conjugation using a rifampicin-resistant spontaneous mutant of Leu. carnosum (Lc-RIF) obtained by mutation and selection on rifampicin-supplemented medium. Leuconostoc lactis and Lc-RIF cultures are then grown to an optical density at 600 nm of 0.5, and the two cultures are then mixed and allowed to stand for 2 hours at 28° C. Aliquots of 200 microliters of the mixed and incubated cultures are then plated in 10⁰, 10⁻¹, 10⁻², 10⁻³, 10⁻⁴ and 10⁻⁵ dilutions on a chemically-defined growth medium for Leuconostoc carnosum (see Example 3) supplemented with 100 micrograms rifampicin and 1% lactose. Under these conditions only Leuconostoc carnosum strains that have received the lactose plasmid by conjugation form colonies. These lactose-positive colonies are re-streaked to single colonies and selected for their level of bacteriocin production.

2) Natural transformation: the Leuconostoc lactis lactose plasmid (pNZ63) is transferred to Leu. carnosum by natural conjugation. A cell- free extract of Leuconostoc lactis is first prepared by sonication. A culture of Leu. carnosum cells is grown under conditions that renders cells naturally competent (see Example 1 and Helmark et al, supra 2004), and the cells are spun down and re-suspended on the chemically-defined growth medium for Leuconostoc carnosum (Example 3) without a source of sugar and mixed with the cell-free extract of Leuconostoc lactis. After a 1 hour incubation at 28° C., the cells are plated in 10⁰, 10⁻¹, 10⁻², 10⁻³, 10⁻⁴ and 10⁻⁵ dilutions on the chemically-defined growth medium for Leuconostoc carnosum supplemented with 1% lactose. Under these conditions only Leuconostoc carnosum strains that have received the lactose plasmid by transformation form colonies. The lactose-positive colonies are re-streaked to single colonies and selected for bacteriocin production level.

3) Transformation: the Leuconostoc lactis lacS and lacLM genes are subcloned and transferred to Leu. carnosum by transformation as follows. The genes lacLM [SEQ ID NO: 12], (encoding a β-galactosidase [SEQ ID NO: 13 and 15) and lacS [SEQ ID NO: 10] (encoding a lactose uptake transporter [SEQ ID NO: 11]), are obtained by means of standard PCR amplification of the respective genes in plasmid DNA of pNZ63 purified from Leuconostoc lactis and the amplified fragments cloned into restriction sites on pCI372. After ligation, the DNA is transformed into Leuconostoc carnosum by standard electroporation as described above. After transformation the cells are plated in 10⁰, 10⁻¹, 10⁻², 10⁻³, 10⁻⁴ and 10⁻⁵ dilutions on the chemically defined growth medium for Leuconostoc carnosum supplemented with 1% lactose. Under these conditions only Leuconostoc carnosum strains that have received the lactose genes inserted in pCI372 form colonies. The colonies are re-streaked to single colonies and analyzed for their level of bacteriocin production.

When the genes lacS [SEQ ID NO: 10] and lacLM [SEQ ID NO: 12] have been transferred to Leu. carnosum by any of the three methods described in this example, the presence of the genes can be verified by standard PCR. The transferred plasmid can be purified by standard plasmid purification for very low-copy number plasmids (for example the “Qiagen very low-copy plasmid purification” kit. Then the existence of the genes on the plasmid can be verified by standard PCR.

### Example 3

### A Chemically Defined Growth Medium for Leuconostoc carnosum

The provision of a chemically defined growth medium for the propagation of Leu. carnosum would greatly facilitate the selection and growth of metabolic mutants, variants or transformants of this micro-organism, including the identification of lactose positive strains. The specific nutritional requirements of Leu. carnosum have not previously been described, however, as a lactic acid bacterium, this organism is expected to have complex requirements, including amino acids, vitamins, salts, micronutrients and nucleobases. The nutritional requirements of other Leuconostoc species described in the literature (Koser, S. A. (1968) Vitamin Requirements of Bacteria and yeasts, Ch. C. Thomas Publisher, Springfield, Ill.; Garvie, E. I. (1986) Leuconostoc. In: Bergey's Manual of Systematic Bacteriology, vol. 2, 9th ed. P. H. A. Sneath, N. S. Mair, Sharpe M. E., and Holt J. G. (eds). Williams & Wilkins, Baltimore, 1071-1075; Dellaglio, F. et al., (1995) The genus Leuconostoc. In: The genera of lactic acid bacteria. Wood, B. J. B., Holzapfel, W. H. (eds). Blackie Academic & Professional, London, 235-278) was used as a basis for designing a chemically defined medium for Leu. carnosum as shown in Table 3.

TABLE 3
A chemically defined growth-medium for Leuconostoc carnosum.
The composition of 1 l of chemically defined medium*
for Leu. Carnosum

| | (g) |
|---|---|
| Amino acids | |
| L-alanine | 0.6 |
| L-glutamate | 0.6 |
| Proline | 0.6 |
| L-serine | 0.6 |
| L-arginine | 0.4 |
| Glycine | 0.4 |
| L-lysine | 0.4 |
| L-phenylalanine | 0.4 |
| L-threonine | 0.4 |
| L-asparagine | 0.25 |
| L-glutamine | 0.25 |
| L-isoleucine | 0.25 |
| L-leucine | 0.25 |
| L-methionine | 0.25 |
| L-tryptophan | 0.25 |
| L-valine | 0.25 |
| L-cystein | 0.2 |
| L-histidine | 0.1 |
| L-tyrosine | 0.1 |
| Nucleobases: | |
| Adenine | 0.02 |
| Guanine | 0.02 |
| Uracil | 0.02 |
| Hypoxanthine | 0.02 |
| Vitamins | |
| Nicotinic acid | 0.002 |
| Thiamin | 0.002 |
| PyridoxinHCl | 0.004 |
| Ca Pantothenate | 0.002 |
| Biotin | 0.01 |
| Folic acid | 0.002 |
| Riboflavin | 0.002 |
| Salts | |
| KH₂PO₄ | 12 |
| K₂HPO₄ | 10 |
| NH₄Cl | 1 |
| MgCl₂ | 0.15 |
| CaCl₂ | 0.015 |
| FeCl₃ | 0.0033 |
| K₂SO₄ | 0.095 |
| Micronutrients: | (nmol) |
| (NH₄)₆Mo₇O₂₄ | 6 |
| H₃BO₄ | 800 |
| CoCl₂ | 60 |
| CuSO₄ | 20 |
| MnCl₂ | 160 |
| ZnSO₄ | 20 |

*The medium must be supplemented with a fermentable carbohydrate to support the growth of Leu. carnosum,

The chemically defined medium includes 19 amino acids, since the amino acid requirements of Leuconostoc species were known to vary significantly. The nucleobase- and vitamin-requirements of hitherto characterized Leuconostoc species include four nucleobases; adenine, guanine, xanthine and uracil and 7 vitamins; nicotinic acid, thiamine, pyridoxine, pantothenate, biotin, folic acid and riboflavin, which were all included in the medium, with the exception that xanthine was replaced by hypoxanthine. A fermentable carbohydrate is added to this medium in order to support the growth of Leu. carnosum.

The growth of Leu. carnosum 4010 in the chemically defined medium supplemented with 1% (w/v) glucose is shown in FIG. 1. In this medium, Leu. carnosum grew to a final OD₆₂₀ of approximately 0.6 and the specific growth rate was 0.44 h⁻¹. When YNB (Yeast Nitrogen Base w/o amino acids and ammonium sulphate, Difco, Detroit) was added to the medium at a concentration of 0.34 g/l, Leu. carnosum grew to a final OD₆₂₀ of 1.0 and the specific growth rate was in the same range (0.43 h⁻¹).

### Example 4

### Selection and Growth of Lactose-Positive Recombinant Leu. carnosum Transformants in Chemically Defined Growth-Medium

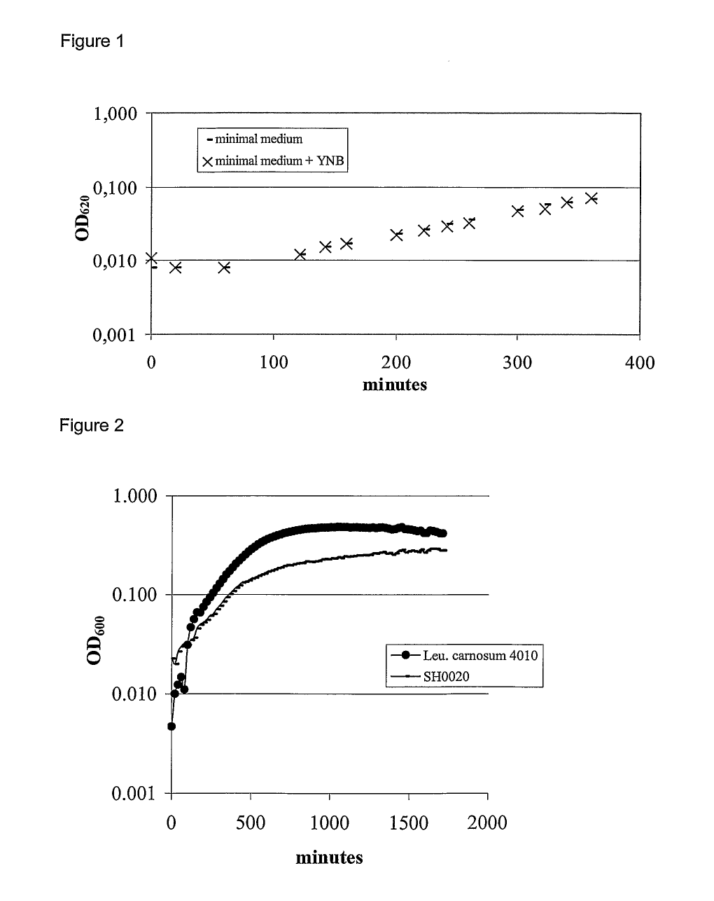

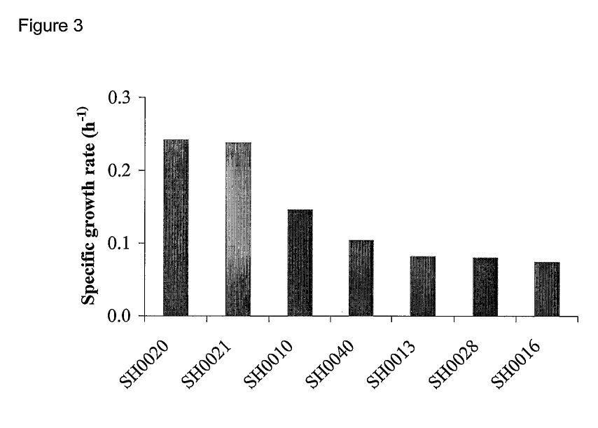

Nine β-galactosidase-positive Leu. carnosum 4010 transformants; SH0010, SH0013, SH0016, SH0020, SH0021, SH0024, SH0028, SH0035 and SH0040, obtained according to Example 1 were inoculated in Leu. carnosum chemically defined medium supplemented with 1% (w/v) lactose as sole fermentable carbohydrate, as defined in Example 2. The strains SH0024 and SH0035 did not grow. The growth of the remaining strains was followed at 27° C. in a Labsystems Bioscreen C from Bie&Berntsen A/S, Rodovre, Denmark by means of OD₆₀₀ measurements (FIG. 2). Due to the different strengths of the randomized promoters, the specific growth rates varied between the strains from 0.074 h⁻¹ (for SH0016) to 0.242 h⁻¹ (for SH0020), as shown in FIG. 3.

### Example 5

### Method of Detecting a Lactose-Positive Leu. carnosum of the Invention

A lactose-positive recombinant Leu. carnosum of the invention is detected by its ability to utilise lactose as sole carbon source, which is tested by screening for growth of a lactose-positive Leu. carnosum colony on a lactose minimal medium. A selective screening medium for detecting said colonies includes the chemically defined growth-medium supplemented with lactose of Example 3 and the steps for performing the screening are as described in Example 4.

The lac+ recombinant Leu. carnosum cells can be distinguished from other cultures in a milk product on the basis of their bacteriocin production. Lac+ recombinant Leu. carnosum strains may be further identified on the basis of their specific phenotypic properties including: vancomycin resistance; growth characteristics of catalase-negative lactic acid bacteria including growth at low temperatures (1-5° C.); production of dextran from sucrose; and an ability to ferment fructose and trehalose apart from sucrose and lactose, but unable to ferment arabinose, arbutin, maltose, raffinose and xylose.

1-37. (canceled)

38. A method for producing a milk product using a recombinant lactose-positive Leuconostoc carnosum strain comprising one or more genes encoding a protein selected from the group consisting of a lactose transporter, a β-galactosidase and β-glucosidase.

39. The method of claim 38, wherein said milk product is selected from the group consisting of cheese, butter milk, sour cream, yoghurt and kefir.

40. The method of claim 38, wherein said strain is a mutant and the product of mutagenesis of one or more native gene of the Leuconostoc carnosum strain.

41. The method of claim 38, wherein said genes are heterologous genes stably inherited in the Leuconostoc carnosum strain.

42. The method of claim 41, wherein said β-galactosidase is a polypeptide having an amino acid sequence that is at least 60% homologous to the amino acid sequence of SEQ ID NO: 3, or a fragment thereof, conferring functional β-galactosidase activity.

43. The method of claim 42, wherein said β-galactosidase has the amino acid sequence of SEQ ID NO: 3.

44. The method of claim 41, wherein said β-galactosidase comprises an L and an M polypeptide having an amino acid sequence that is at least 60% homologous to the amino acid sequence of SEQ ID NO: 13 and 15, respectively, or a fragment thereof, and confers functional β-galactosidase activity.

45. The method of claim 41, wherein said β-galactosidase comprises an L and an M polypeptide having the amino acid sequence of SEQ ID NO: 13 and 15, respectively.

46. The method of claim 42, wherein said β-galactosidase is encoded by a nucleic acid molecule that hybridizes to a nucleic acid molecule with a nucleotide sequence consisting of SEQ ID NO: 1 or 4, under washing stringency conditions of no less than 1×SSC at 65° C.

47. The method of claim 46, wherein said β-galactosidase encoding nucleic acid molecule has the nucleotide sequence of SEQ ID NO: 1 or 4.

48. The method of claim 44, wherein said L and M polypeptide are encoded by a nucleic acid molecule that hybridizes to a nucleic acid molecule with a nucleotide sequence consisting of SEQ ID NO: 12, under washing stringency conditions of no less than 1×SSC at 65° C.

49. The method of claim 48, wherein said L and M polypeptide encoding nucleic acid molecule has the nucleotide sequence of SEQ ID NO: 12.

50. The method of claim 41, wherein said lactose transporter is a polypeptide having an amino acid sequence that is at least 60% homologous to the amino acid sequence of SEQ ID NO: 2, or a fragment thereof, conferring functional lactose transporter activity.

51. The method of claim 50, wherein said lactose transporter has the amino acid sequence of SEQ ID NO: 2.

52. The method of claim 41, wherein said lactose transporter is a polypeptide having an amino acid sequence that is at least 60% homologous to the amino acid sequence of SEQ ID NO: 11, or a fragment thereof, conferring functional lactose transporter activity.

53. The method of claim 52, wherein said lactose transporter has the amino acid sequence of SEQ ID NO: 11.

54. The method of claim 50, wherein said lactose transporter is encoded by a nucleic acid molecule that hybridizes to a nucleic acid molecule with a nucleotide sequence consisting of SEQ ID NO: 1 or 6, under washing stringency conditions of no less than 1×SSC at 65° C.

55. The method of claim 54, wherein said lactose transporter encoding nucleic acid molecule has the nucleotide sequence of SEQ ID NO: 1 or 6.

56. The method of claim 52, wherein said lactose transporter is encoded by a nucleic acid molecule that hybridizes to a nucleic acid molecule with a nucleotide sequence consisting of SEQ ID NO: 10, under washing stringency conditions of no less than 1×SSC at 65° C.

57. The method of claim 56, wherein said lactose transporter encoding nucleic acid molecule has the nucleotide sequence of SEQ ID NO: 10

58. The method of claim 46, wherein at least one nucleic acid molecule corresponding to said genes which encode a lactose transporter or a β-galactosidase is harbored on a self-replicating plasmid.

59. The method of claim 58, wherein said plasmid is selected from the group consisting of pC1372, pGhost⁺8, and pC13340.

60. The method of claim 46, wherein at least one nucleic acid molecule corresponding to said genes which encode a lactose transporter or a β-galactosidase is integrated into the genome of said strain, said genome including a bacterial chromosome and/or a native plasmid.

61. The method of claim 46, wherein at least one nucleic acid molecule corresponding to said genes which encode a lactose transporter or a β-galactosidase is comprised within an operon.

62. The method of claim 61, wherein said nucleic acid molecule or operon is operably linked to a homologous or heterologous promoter.

63. The method of claim 38, wherein said strain is selected on a chemically defined growth-medium comprising amino acids, nucleotide bases, vitamins, salts and micronutrients supplemented with lactose as the sole fermentable sugar.

64. The method of claim 58, wherein said strain is sensitive to an antibiotic selected from the group consisting of penicillin, chloramphenicol, tetracycline, erythromycine and kanamycin.

65. An isolated, recombinant lactose-positive Leuconostoc carnosum strain comprising one or more genes encoding a protein selected from the group consisting of a lactose transporter, a β-galactosidase and a β-glucosidase,
wherein said genes are stably inherited in said Leuconostoc carnosum strain, and
wherein said lactose transporter is encoded by a nucleic acid molecule that hybridizes to a nucleic acid molecule with a nucleotide sequence consisting of SEQ ID NO: 1, 6 or 10, under washing stringency conditions of no less than 1×SSC at 65° C., and
wherein said β-galactosidase is encoded by a nucleic acid molecule that hybridizes to a nucleic acid molecule with a nucleotide sequence consisting of SEQ ID NO: 1, 4 or 12, under washing stringency conditions of no less than 1×SSC at 65° C.

66. The recombinant lactose-positive Leuconostoc carnosum strain of claim 65, wherein said nucleic acid molecule encoding said lactose transporter has the nucleotide sequence of SEQ ID NO: 1, 6 or 10, and
wherein said nucleic acid molecule encoding said β-galactosidase has the nucleotide sequence of SEQ ID NO: 1, 4 or 12.

67. The recombinant lactose-positive Leuconostoc carnosum strain of claim 65, wherein said lactose transporter is a polypeptide having an amino acid sequence that is at least 60% homologous to the amino acid sequence of SEQ ID NO: 2, 7 or 11, or a fragment thereof, conferring functional lactose transporter activity, and
wherein said β-galactosidase is a polypeptide having an amino acid sequence that is at least 60% homologous to the amino acid sequence of SEQ ID NO: 3, 5 or 13+15, or a fragment thereof, conferring functional β-galactosidase activity.

68. The recombinant lactose-positive Leuconostoc carnosum strain of claim 67, wherein said lactose transporter has the amino acid sequence of SEQ ID NO: 2, 7 or 11, and
wherein said β-galactosidase has the amino acid sequence of SEQ ID NO: 3, 5 or 13+15.

69. A starter culture for use in the production of a milk product, comprising a recombinant, lactose-positive Leuconostoc carnosum strain, wherein said strain comprises one or more genes encoding a protein selected from the group consisting of a lactose transporter, a β-galactosidase and a β-glucosidase, and
one or more bacterial or fungal strains selected from the group consisting of the species Lactobacillus, Lactococcus, Leuconostoc, Streptococcus Penicillium and Geotrichum.

70. A method for producing a milk product using the starter culture of claim 69, wherein said milk product is selected from the group consisting of cheese, butter milk, sour cream, yoghurt and kefir.

71. A milk product comprising the starter culture of claim 69.

72. The milk product of claim 71, wherein said milk product is selected from the group consisting of cheese, butter milk, sour cream, yoghurt and kefir.

73. A method for producing a milk product comprising the steps of:
(a) adding the starter culture of claim 69 to a volume of milk,
(b) incubating the product of (a) to form a cheese milk,
(c) coagulating the product of (b) to form milk curds, and
(d) separating the milk curds from the product of (c).

74. A method for constructing the recombinant Leuconostoc carnosum strain of claim 65, comprising the steps of:
(a) transforming Leuconostoc carnosum with at least one nucleic acid molecule, each said nucleic acid molecule encoding a lactose transporter or β-galactosidase, and
(b) selecting transformed cells of Leuconostoc carnosum characterized by the ability to grow on lactose as sole carbon source.

75. A milk product produced according to the method of claim 73.

76. A milk product comprising the Leuconostoc carnosum strain of claim 65.

77. The milk product of claim 76, wherein said milk product is selected from the group consisting of cheese, butter milk, sour cream, yoghurt and kefir.

78. A method for producing a milk product comprising the steps of:
(a) adding a cell culture comprising cells of a Leuconostoc carnosum strain of claim 67 to a volume of milk,
(b) incubating the product of (a) to form a cheese milk,
(c) coagulating the product of (b) to form milk curds, and
(d) separating the milk curds from the product of (c).

79. A milk product produced according to the method of claim 78.


SEQUENCE LISTING (15 sequences)

SEQ ID NO: 1 - DNA, 5206 bp, Streptococcus thermophilus
  misc_structure (1)..(15)
  promoter (16)..(94)
  misc_feature (18)..(22): n is a, c, g, or t
acgactagtggatccatnnnnnagtttattcttgacannnnnnnnnnnnnntgrtataatnnnnaagtaataaaatattcggaggaattttgaaatggaaaaatctaaaggtcagatgaagtctcgtttatcctacgcagctggtgcttttggtaacgacgtcttctatgcaaccttgtcaacatactttatcatgtttgtgacaactcacttgtttaacacaggtgatccaaagcaaaatagtcactacgtactattaatcactaacattatctctattttgcgtatcttggaagtatttatcgatccattgatcggtaatatgattgataacactaatactaagtatggtaaattcaaaccatgggtagttggtggtggtatcatcagttctatcaccttgttgtttctcttaaccgatataggtggtttgaataaaacaaatcctttcttgtatcttgtactttttggaattatctaccttgtaatggatgtcttctactcgattaaagatatcggtttctggtcaatgattcctgccttgtctcttgatagtcacgaacgtgaaaaaatggcaacttttgcccgtattggttcgacgattggtgccaatattgtaggtgttgccatcatgccaatcgatttgttattctctatgacgcacaatagtggctctggagataaatctggatggttctggtatgcatttatcgtagatctcaatggtgtgattacatcaattgctgttggtattggtacacgtgaagttgagtcaaaaattcatgataataacgaaaaaactagccttaaacaagtctttaaggttcttggtcaaaacgaccaaatgatgtggttatctcttggatattggttctatggtcttggtattaatacacttaatgctcttcaactttattatttcacatttatccttggtgattcaggtaaatactcaattctttacggattgaatacagttgttggtttggtttcagtttcactcttccctaccctagctgataaattcaaccgtaaacgtttgttctacggatgtattgcagtaatgctcgggggtatcggaatatttagtattgcaggtacatcacttccaataatcttgactgcagctgaactcttcttcattccacaacctcttgtgttccttgttgtctttatgattatctctgactcagtagaatatggtcaatggaaaacgggacaccgtgatgaatcacttactttgtcagttcgtccacttattgataaacttggtggtgcgatgtcaaactggcttgtttctacatttgccgtagctgccggtatgacaacaggtgcctcagcatcaacaattacaacacatcaacagtttatctttaagcttggcatgtttgctttcccagcagcaacaatgcttatcggtgccttcattgttgctcgtaaaatcactttgactgaagcacgtcacgctaaaattgttgaagaattggaacatcgctttagcgtagcaacttctgaaaatgaagttaaagctaacgtcgtatctcttgtaacccctacaactggttatttggttgatctctcaagtgttaatgatgaacactttgcttcaggtagcatgggtaaaggtttcgccattaaacctactgatggagctgtctttgcaccaattagtggtaccattcgtcaaattcttcctactcgccatgcagttggtattgaaagtgaagatggtgtcattgttcttatccacgttggcatcggaacagttaaacttaatggtgaaggattcattagttacgtagaacaaggtgatcatgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataatctatgaacatgactgaaaaaattcaaacttatttaaacgatccaaagattgttagcgttaatactgttgatgctcactcagatcataagtattttgaatctcttgaagaattttctgaaggggagatgaagttaagacaatctcttaatggaaaatggaaaattcactatgctcagaatacaaatcaggttttaaaagacttttataaaacagaatttgatgaaactgatttgaatttcatcaatgtaccaggtcatttagagcttcaaggttttggttctccacaatatgtgaatacccaatatccttgggatggtaaagaattccttcgtccacctcaagttcctcaagaatcaaatgctgttgcatcatacgttaaacattttaccttgaatgatgcattaaaagataaaaaagtatttatctcattccaaggggttgctacttccatctttgtatgggtcaatggtaacttcgtaggatacagtgaagattcatttacacctagtgaatttgaaattagtgattaccttgttgaaggtgataacaagttggcggtagctgtttatcgttactctacagcaagctggttggaagaccaagacttctggagactttacggtatttttagagatgtttacttgtatgctattccaaaagttcacgttcaagatctctttgttaagggagattatgattaccaaacaaaagcaggtcaattggatattgatttgaagactgttggtgattatgaagacaagaagattaaatatgttctttcagattatgaaggcatcgttacagaaggtgatgcatctgttaatggtgacggtgaactatctgtaagtcttgaaaatcttaaaatcaaaccttggagtgctgaaagtcctaaactttacgatttgatccttcatgttttggatgatgaccaagttgttgaagtcgttccagttaaagttggatttagacgctttgaaattaaagataaacttatgcttttgaatggtaagagaattgtctttaaaggggttaacagacacgaatttaacgctagaacaggacgttgtatcactgaagaagatatgctttgggatatcaaagtgatgaaacaacataacatcaatgctgttcgtacttcacactatcctaaccatacacgttggtatgaattgtgtgatgaatatggactttatgttatcgatgaagccaaccttgaaacacacggtacatggcaaaaacttggtctatgcgaaccttcatggaatatcccagctagtgaaccagaatggttgcctgcttgtttggatcgtgccaataacatgttccaacgcgataagaaccacgctagtgttatcatttggtcttgtggtaatgaatcatatgctggtaaagatattgctgacatggctgattacttccgtagtgttgacaatactcgtccagttcactatgaaggtgttgcatggtgtcgtgagtttgattacattacagacatcgaaagtcgtatgtatgcgaaaccagctgatatcgaagaatacctcacaactggtaaactagttgatctttcaagcgttagtgataaacactttgcttcaggtaacctaactaacaaacctcaaaaaccttatatttcatgtgaatacatgcacacaatgggtaactctggtggtggattgcaactctacactgacttagagaaatatccagaataccaaggtggatttatttgggacttcattgaccaagctatttacaaaacacttccaaatggtagcgaattcctatcatatggtggtgactggcatgatagaccttctgactacgaattttgtggaaatggtatcgtctttgcagatcgtaccctaactccaaaacttcaaacagttaaacatctttactctaatattaagattgctgttgatgaaaaatcagtaactatcaagaatgataatctcttcgaagatctttctgcttatactttcctagctagagtttacgaagatggtagaaaagttagtgaaagtgaatatcactttgatgttaaaccaggcgaagaagcaacattcccagttaactttgtagtcgaggcttcaaattctgaacaaatttacgaagttgcttgtgttctgagggaagcaactgaatgggctcctaaaggtcatgaaattgttcgtggtcaatatgttgttgaaaagattagcactgaaacaccagttaaagcacctttgaatgttgttgaaggcgacttcaacatcggtattcaaggacaaaacttctcaatcttgctttcacgtgcacaaaatactttagtatctgctaagtataatggtgttgaattcattgagaaaggtcctaaacttagcttcactcgtgcttacactgacaacgatcgtggtgctggatatccattcgaaatggcaggctggaaggttgctggaaactatagtaaagttacagatactcaaattcaaatcgaagacgactctgttaaagtgacttatgttcatgaattgccaggcttgtctgatgtcgaagttaaggtaacttatcaagttgattacaagggtcgaatctttgttactgcaaactatgatggtaaagcaggtttgccaaacttccctgaatttggtttagaatttgctatcggttcacaatttacaaaccttagctattatggatacggtgcagaagaaagctaccgtgataaacttcctggtgcctatcttggtcgatatgaaacatctgttgaaaagacatttgctccatatctaatgccacaagaatctggtaatcactatggtactcgtgaattcacagtatctgatgataaccataatggtcttaaattcaccgcacttaataaagcattcgaattcagtgctttgcgtaacactactgaacaaattgaaaatgctcgtcaacaatatgagttgcaagaatctgatgctacatggattaaagtgcttgctgctcaaatgggtgtaggtggtgacgacacatggggtgctccagttcatgacgaattcttgcttagctcagcagatagctatcaattaagcttcatgattgaaccactaaattaggtagttattaagaatctagttgtacattatttgataattataaaagagaagctttggaggcttctcttttcatgttttaaaggagattaaatcattagtgtgttagtatcttcctcgagtacc

SEQ ID NO: 2 - protein, 634 aa, Streptococcus thermophilus
MEKSKGQMKSRLSYAAGAFGNDVFYATLSTYFIMFVTTHLFNTGDPKQNSHYVLLITNIISILRILEVFIDPLIGNMIDNTNTKYGKFKPWVVGGGIISSITLLFLLTDIGGLNKTNPFLYLVLFGIIYLVMDVFYSIKDIGFWSMIPALSLDSHEREKMATFARIGSTIGANIVGVAIMPIDLLFSMTHNSGSGDKSGWFWYAFIVDLNGVITSIAVGIGTREVESKIHDNNEKTSLKQVFKVLGQNDQMMWLSLGYWFYGLGINTLNALQLYYFTFILGDSGKYSILYGLNTVVGLVSVSLFPTLADKFNRKRLFYGCIAVMLGGIGIFSIAGTSLPIILTAAELFFIPQPLVFLVVFMIISDSVEYGQWKTGHRDESLTLSVRPLIDKLGGAMSNWLVSTFAVAAGMTTGASASTITTHQQFIFKLGMFAFPAATMLIGAFIVARKITLTEARHAKIVEELEHRFSVATSENEVKANVVSLVTPTTGYLVDLSSVNDEHFASGSMGKGFAIKPTDGAVFAPISGTIRQILPTRHAVGIESEDGVIVLIHVGIGTVKLNGEGFISYVEQGDHVEVGQKLLEFWSPIIEKNGLDDTVLVTVTNSEKFSAFHLEQKVGEKVEALSEVITFKKGE

SEQ ID NO: 3 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNHTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNTTEQIENARQQYELQESDATWIKVLAAQMGVGGDDTWGAPVHDEFLLSSADSYQLSFMIEPLN

SEQ ID NO: 4 - DNA, 3081 bp, Streptococcus thermophilus
  CDS (1)..(3078): LacZ CDS
atgaacatgactgaaaaaattcaaacttatttaaacgatccaaagattgttagcgttaatactgttgatgctcactcagatcataagtattttgaatctcttgaagaattttctgaaggggagatgaagttaagacaatctcttaatggaaaatggaaaattcactatgctcagaatacaaatcaggttttaaaagacttttataaaacagaatttgatgaaactgatttgaatttcatcaatgtaccaggtcatttagagcttcaaggttttggttctccacaatatgtgaatacccaatatccttgggatggtaaagaattccttcgtccacctcaagttcctcaagaatcaaatgctgttgcatcatacgttaaacattttaccttgaatgatgcattaaaagataaaaaagtatttatctcattccaaggggttgctacttccatctttgtatgggtcaatggtaacttcgtaggatacagtgaagattcatttacacctagtgaatttgaaattagtgattaccttgttgaaggtgataacaagttggcggtagctgtttatcgttactctacagcaagctggttggaagaccaagacttctggagactttacggtatttttagagatgtttacttgtatgctattccaaaagttcacgttcaagatctctttgttaagggagattatgattaccaaacaaaagcaggtcaattggatattgatttgaagactgttggtgattatgaagacaagaagattaaatatgttctttcagattatgaaggcatcgttacagaaggtgatgcatctgttaatggtgacggtgaactatctgtaagtcttgaaaatcttaaaatcaaaccttggagtgctgaaagtcctaaactttacgatttgatccttcatgttttggatgatgaccaagttgttgaagtcgttccagttaaagttggatttagacgctttgaaattaaagataaacttatgcttttgaatggtaagagaattgtctttaaaggggttaacagacacgaatttaacgctagaacaggacgttgtatcactgaagaagatatgctttgggatatcaaagtgatgaaacaacataacatcaatgctgttcgtacttcacactatcctaaccatacacgttggtatgaattgtgtgatgaatatggactttatgttatcgatgaagccaaccttgaaacacacggtacatggcaaaaacttggtctatgcgaaccttcatggaatatcccagctagtgaaccagaatggttgcctgcttgtttggatcgtgccaataacatgttccaacgcgataagaaccacgctagtgttatcatttggtcttgtggtaatgaatcatatgctggtaaagatattgctgacatggctgattacttccgtagtgttgacaatactcgtccagttcactatgaaggtgttgcatggtgtcgtgagtttgattacattacagacatcgaaagtcgtatgtatgcgaaaccagctgatatcgaagaatacctcacaactggtaaactagttgatctttcaagcgttagtgataaacactttgcttcaggtaacctaactaacaaacctcaaaaaccttatatttcatgtgaatacatgcacacaatgggtaactctggtggtggattgcaactctacactgacttagagaaatatccagaataccaaggtggatttatttgggacttcattgaccaagctatttacaaaacacttccaaatggtagcgaattcctatcatatggtggtgactggcatgatagaccttctgactacgaattttgtggaaatggtatcgtctttgcagatcgtaccctaactccaaaacttcaaacagttaaacatctttactctaatattaagattgctgttgatgaaaaatcagtaactatcaagaatgataatctcttcgaagatctttctgcttatactttcctagctagagtttacgaagatggtagaaaagttagtgaaagtgaatatcactttgatgttaaaccaggcgaagaagcaacattcccagttaactttgtagtcgaggcttcaaattctgaacaaatttacgaagttgcttgtgttctgagggaagcaactgaatgggctcctaaaggtcatgaaattgttcgtggtcaatatgttgttgaaaagattagcactgaaacaccagttaaagcacctttgaatgttgttgaaggcgacttcaacatcggtattcaaggacaaaacttctcaatcttgctttcacgtgcacaaaatactttagtatctgctaagtataatggtgttgaattcattgagaaaggtcctaaacttagcttcactcgtgcttacactgacaacgatcgtggtgctggatatccattcgaaatggcaggctggaaggttgctggaaactatagtaaagttacagatactcaaattcaaatcgaagacgactctgttaaagtgacttatgttcatgaattgccaggcttgtctgatgtcgaagttaaggtaacttatcaagttgattacaagggtcgaatctttgttactgcaaactatgatggtaaagcaggtttgccaaacttccctgaatttggtttagaatttgctatcggttcacaatttacaaaccttagctattatggatacggtgcagaagaaagctaccgtgataaacttcctggtgcctatcttggtcgatatgaaacatctgttgaaaagacatttgctccatatctaatgccacaagaatctggtaatcactatggtactcgtgaattcacagtatctgatgataaccataatggtcttaaattcaccgcacttaataaagcattcgaattcagtgctttgcgtaacactactgaacaaattgaaaatgctcgtcaacaatatgagttgcaagaatctgatgctacatggattaaagtgcttgctgctcaaatgggtgtaggtggtgacgacacatggggtgctccagttcatgacgaattcttgcttagctcagcagatagctatcaattaagcttcatgattgaaccactaaattag

SEQ ID NO: 5 - protein, 1026 aa, Streptococcus thermophilus; identical to SEQ ID NO: 3

SEQ ID NO: 6 - DNA, 1905 bp, Streptococcus thermophilus
  CDS (1)..(1902): LacS CDS
atggaaaaatctaaaggtcagatgaagtctcgtttatcctacgcagctggtgcttttggtaacgacgtcttctatgcaaccttgtcaacatactttatcatgtttgtgacaactcacttgtttaacacaggtgatccaaagcaaaatagtcactacgtactattaatcactaacattatctctattttgcgtatcttggaagtatttatcgatccattgatcggtaatatgattgataacactaatactaagtatggtaaattcaaaccatgggtagttggtggtggtatcatcagttctatcaccttgttgtttctcttaaccgatataggtggtttgaataaaacaaatcctttcttgtatcttgtactttttggaattatctaccttgtaatggatgtcttctactcgattaaagatatcggtttctggtcaatgattcctgccttgtctcttgatagtcacgaacgtgaaaaaatggcaacttttgcccgtattggttcgacgattggtgccaatattgtaggtgttgccatcatgccaatcgatttgttattctctatgacgcacaatagtggctctggagataaatctggatggttctggtatgcatttatcgtagatctcaatggtgtgattacatcaattgctgttggtattggtacacgtgaagttgagtcaaaaattcatgataataacgaaaaaactagccttaaacaagtctttaaggttcttggtcaaaacgaccaaatgatgtggttatctcttggatattggttctatggtcttggtattaatacacttaatgctcttcaactttattatttcacatttatccttggtgattcaggtaaatactcaattctttacggattgaatacagttgttggtttggtttcagtttcactcttccctaccctagctgataaattcaaccgtaaacgtttgttctacggatgtattgcagtaatgctcgggggtatcggaatatttagtattgcaggtacatcacttccaataatcttgactgcagctgaactcttcttcattccacaacctcttgtgttccttgttgtctttatgattatctctgactcagtagaatatggtcaatggaaaacgggacaccgtgatgaatcacttactttgtcagttcgtccacttattgataaacttggtggtgcgatgtcaaactggcttgtttctacatttgccgtagctgccggtatgacaacaggtgcctcagcatcaacaattacaacacatcaacagtttatctttaagcttggcatgtttgctttcccagcagcaacaatgcttatcggtgccttcattgttgctcgtaaaatcactttgactgaagcacgtcacgctaaaattgttgaagaattggaacatcgctttagcgtagcaacttctgaaaatgaagttaaagctaacgtcgtatctcttgtaacccctacaactggttatttggttgatctctcaagtgttaatgatgaacactttgcttcaggtagcatgggtaaaggtttcgccattaaacctactgatggagctgtctttgcaccaattagtggtaccattcgtcaaattcttcctactcgccatgcagttggtattgaaagtgaagatggtgtcattgttcttatccacgttggcatcggaacagttaaacttaatggtgaaggattcattagttacgtagaacaaggtgatcatgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataa

SEQ ID NO: 7 - protein, 634 aa, Streptococcus thermophilus; identical to SEQ ID NO: 2

SEQ ID NO: 8 - DNA, 115 bp, Artificial
  forward synthetic primer
acgactagtggatccatnnnnnagtttattcttgacannnnnnnnnnnnnntgrtataatnnnnaagtaataaaatattcggaggaattttgaaatggaaaaatctaaaggtcag

SEQ ID NO: 9 - DNA, 31 bp, Streptococous thermophilis
ggtactcgaggaagatactaacacactaatg

SEQ ID NO: 10 - DNA, 2130 bp, Leuconostoc lactis
  5′UTR (1)..(110)
  CDS (111)..(2030)
  3′UTR (2031)..(2130)
gatagggttgaacaacaaaacgggaatattattttagctactccaagtgcactacattggaaccatatcaggttatcgtagcaacaaaggactgatttaggagaaagaatatgaaagacataactaaacagaagttttctcgaaataagctagtagaaatgatttcttttgcacttggtaatctagggcacgctgcattctatggtgcattaagcacttacttcatcgtttatgtcacgagtggtatgtttgatgggttaccgcaatcggtagcaaacaagttaattggtttaattacggctcttgtagtaattattcgtttggccgaagtaattattgatccgattcttggaaatattgtagataatacaaaaacacgatggggaaaatttaaaccttggcaagtaattggcgcggttgttagttcagtgttattagttgtaatatttactggaatttttgggttggctcatattaattggattgcttttgcgatcgttttcacagttttatttattttactagatattttttattcatttgcagatgttgcctattggggaatggtgccggctatatctgaagacagtaaagagagaggaatttttacttccttagggagttttactggttctattggttggaatggtcttacgatgattgttgttccggtcaccacatacttcacttttatcgcaactggaaaacacgaacaagggccttcggggtggtttggattttcaatagttgtttccattgtagccgttttatcagctttggcagtagcctttggaactaaagaaaaagataacttgattcgaaatgctgcaacaaaaaaaacaagcattaaagatgtattttcaggtattattcataatgaccaaattttatggatcagcttagcatatctaatgtattcactagcgtatgttgtgactaatggtgttttattttacttctttaagtttgtactaggaaaaccaaacgaattttggattgctggtgctattgcaacagtaatagggttctctactgctccgttgtatcccgttctaaataagtttatcaccaggaaagtgttatttagtattgggcaaatggcaatgatcctatcatatctgttttttatattcggaaaaacaaatatgatgatggttacaataggacttattttgtttaattttacttttgcgcaactggttgttgtcttatcactaactgactctattgaatacggacaattaaaaaatggtaatcgcaacgaagctgttgtcttggcggttcgacctatgttagataagataactggtgctttttcaaacggactcgtcggagcaattgcaattacggcgggcatgactggatcagctaccgcaggagatattagtgcatcaaaaattaatacctttgaaatttatgctttttacacaccacttcttttttctattctagctttggttatattcttgtggaaagttaaaattaccgagaaaaaacatgccgaaatcgttattgaactggaaaaaactttatcaagtggcgctaaaaaagctaatacttctgaagtgaatgttgaacttgaagaaatatttgcacctgcctcaggacaaaaaaagctacttaatgaggttgatggcaacacacttactggtattggttttgctattgatccggaagaaggtaatttatttgcaccatttgatggcaaagtcgactttactttttctacaaaacatgttttgggtgttgtatctaacaacggattaaaagcaattattcatgtaggaataggcactatcaatatgcgaggtgcggggtttgtgtcacattatgtggacggccaattgttcaaaaagggagatcttttgatgacctttgataagaaattaattacgaaaaacgggtatcaagatgacattattatgtactttacccaaccagaaaacattatagatgttcaacaaattgataatcgtgtcgtcaaacaaggggagaagatagcaaaattaacattcaggagtgaaagataacttaaaggtgggccggaaagtctgaacttaaaaagagattggtgggtagtaatcactacaatcgcaataactagttttctttcattttcagtatcggctt

SEQ ID NO: 11 - protein, 639 aa, Leuconostoc lactis
MKDITKQKFSRNKLVEMISFALGNLGHAAFYGALSTYFIVYVTSGMFDGLPQSVANKLIGLITALVVIIRLAEVIIDPILGNIVDNTKTRWGKFKPWQVIGAVVSSVLLVVIFTGIFGLAHINWIAFAIVFTVLFILLDIFYSFADVAYWGMVPAISEDSKERGIFTSLGSFTGSIGWNGLTMIVVPVTTYFTFIATGKHEQGPSGWFGFSIVVSIVAVLSALAVAFGTKEKDNLIRNAATKKTSIKDVFSGIIHNDQILWISLAYLMYSLAYVVTNGVLFYFFKFVLGKPNEFWIAGAIATVIGFSTAPLYPVLNKFITRKVLFSIGQMAMILSYLFFIFGKTNMMMVTIGLILFNFTFAQLVVVLSLTDSIEYGQLKNGNRNEAVVLAVRPMLDKITGAFSNGLVGAIAITAGMTGSATAGDISASKINTFEIYAFYTPLLFSILALVIFLWKVKITEKKHAEIVIELEKTLSSGAKKANTSEVNVELEEIFAPASGQKKLLNEVDGNTLTGIGFAIDPEEGNLFAPFDGKVDFTFSTKHVLGVVSNNGLKAIIHVGIGTINMRGAGFVSHYVDGQLFKKGDLLMTFDKKLITKNGYQDDIIMYFTQPENIIDVQQIDNRVVKQGEKIAKLTFRSER

SEQ ID NO: 12 - DNA, 3195 bp, Leuconostoc lactis
  5′UTR (1)..(276)
  CDS (277)..(2157): LacL CDS
gtcgaccgaaggtccgtattcatcggatagatcagtatatgaacggccttgattgtgcatctcaacaagagactgcttaaattcttttttgtagcgaatcatattaaaaaatgtctcctatgcgattagtttacagggcagaagaaaatctgtccgtaaatctagcataagagcatttagttaaaatttaactaaatgacatatataaatttaatattttgttttataataattgtaagcgttttttatttatgtaactttgaaaggatcttcctcatgcaagctaatcttcaatggttagatgacccagaagtcttccgggtcaaccaattacctgcacatagtgatcaccattattatcacgacacagcagaattcaaaacgggtagtcgcttcatcaagagtctcaatggcgcttggcgttttaacttcgccaagacaccggctgaacgcccagttgatttttatcaacccgatttcgatgcaaccgactttgatacgattcaagttcccggtcatattgaactagccggctatggtcaaattcaatacattaacacgctatacccatgggaaggtaaaatttaccgtcgcccaccgtataccctcaatcaagatcaattaacaccaggcctattcagcgacgctgcggacaacaccgtcggctcgtacctcaaaaccttcgatctcgacgatgtttttaaagggcaacgtattatcattcagttccaaggggtagaagaagccctgtacgtctggttaaatggccattttattggctactctgaagatagtttcaccccttcagaatttgatttgacgccgtatattcaggaccaaggtaacgttttagcggttcgggtctacaaacacagtactgctgcctttattgaagaccaagatatgttccgtttctctggtattttccgtgacgtcaatatactggcggagcctgctagccatattactgatttggacatccgaccagttccaaatgccaatctcaaaagtggtgagctcaacatcactactaaagtaaccggcgaaccagccactttagcgctgaccgttaaagaccatgacgggcgagtactgacgagtcaaacgcaaaccggtagtggcagtgtaacctttgatactatgttattcgaccaactgcacttgtggtcaccacaaacgccgtatctctatcaattgacaattgaagtttacgatgctgatcaccaactcttggaagtcgtcccatatcagtttgggttccggacggtcgagctgcgcgatgacaaagtcatttacgtcaacaataaacggttggtgatcaacggggttaaccggcacgaatggaacgcccacaccggtcgcgttatcagtatggctgatatgcgcgctgatatccaaaccatgttagctaacaatatcaatgccgatcggacctgccattatcctgatcaattaccttggtatcaattatgtgacgaggccggtatctacctaatggccgaaaccaacctcgaatcgcacgggtcatggcaaaagatgggggctatcgagccttcttacaatgttcctggcgataatccacactggccagcagcggtgatcgaccgggcccgttcaaactacgaatggtttaaaaaccacccatcaatcattttttggtcacttggcaatgaatcgtatgctggcgaagatatcgcggcgatgcaggctttttataaagaacacgatgattcacgactcgtccactacgaaggcgttttctacacaccagaattaaaagatcgcatttctgatgttgaaagtcggatgtacgaaaagccccaaaatattgtagcttacttggaagataacccaaccaaacctttcctaaattgtgaatatatgcatgacatggggaattctctgggcggtatgcaatcatataatgatttgatcgacaagtatccaatgtatcaaggtggctttatttgggactttattgatcaagcactcttcgttcatgacccaatcaccgaccaagacgtgctccggtatggcggtgatttcgacgaacgccactccgattatgcattctccggtaacggcttaatgtttgccgaccggacaccaaaaccagcaatgcaagaggtgaaatattattatggcttacacaaataatcaactacacgttatttacggcgacgggagcttaggactacagggggctaatttccactacctctttagctacgaacgtgggggacttgaatcactcgtcgtcaacgataaagagtggctctatcgtacacccacgcccatgttttggcgggcgacaaccgataatgatcacggtagcggcttttcagtcaaatccgcacagtggtacgcggccgataagttctcaacttgtcaagatatcgaattgacggttgacgaccaaccagtcacaccgttaccaatcgcgccactcaataacaaatacacggatcacgaaatcgccacgaaagtctctctggcttaccacttcgttaccacgaccgttcctagtaccatcgtcacagtgacttatacggtgacagcagacggtcagatcaatatcgccacccattatagcggtcagtctgatttgccagagctacccgcatttggtctgcggtttatcatgccaactaccgcgaccggcttcgactataccggtttgtccggtgagacttatcctgaccggctggctggcgcaacacacgggcaattccacgttgacagtctgccagtcacaccatacttggtcccacaagaatgcggcatgcacatgcaaactgaacaagtgacagtaacgcgatcaacaacacaaaataacgctgaccacgacaacacaccattcagtttaacatttagccaaaccgatgcaccattcgccttcagctgccttccctataccgctgctgaactagaaaacgcaacacacatggaagaattaccattagcacggcgaacggtcttatcaatctacggtgccgttcgtggggtcggtggcattgacagttggggaacggacgtagaagccccatatcatatcctcgctaatcaagacattgacttcagctttaacattcatttctaaaaattaatttgatttcaaaagaatcgctccggcagttatttgccagagcgttcttttaatatgttttatggctgagctttagtcctttgaagtga

SEQ ID NO: 13 - protein, 626 aa, Leuconostoc lactis
MQANLQWLDDPEVFRVNQLPAHSDHHYYHDTAEFKTGSRFIKSLNGAWRFNFAKTPAERPVDFYQPDFDATDFDTIQVPGHIELAGYGQIQYINTLYPWEGKIYRRPPYTLNQDQLTPGLFSDAADNTVGSYLKTFDLDDVFKGQRIIIQFQGVEEALYVWLNGHFIGYSEDSFTPSEFDLTPYIQDQGNVLAVRVYKHSTAAFIEDQDMFRFSGIFRDVNILAEPASHITDLDIRPVPNANLKSGELNITTKVTGEPATLALTVKDHDGRVLTSQTQTGSGSVTFDTMLFDQLHLWSPQTPYLYQLTIEVYDADHQLLEVVPYQFGFRTVELRDDKVIYVNNKRLVINGVNRHEWNAHTGRVISMADMRADIQTMLANNINADRTCHYPDQLPWYQLCDEAGIYLMAETNLESHGSWQKMGAIEPSYNVPGDNPHWPAAVIDRARSNYEWFKNHPSIIFWSLGNESYAGEDIAAMQAFYKEHDDSRLVHYEGVFYTPELKDRISDVESRMYEKPQNIVAYLEDNPTKPFLNCEYMHDMGNSLGGMQSYNDLIDKYPMYQGGFIWDFIDQALFVHDPITDQDVLRYGGDFDERHSDYAFSGNGLMFADRTPKPAMQEVKYYYGLHK

SEQ ID NO: 14 - DNA, 3195 bp, Leuconostoc lactis; identical to SEQ ID NO: 12
  5′UTR (1)..(2140)
  CDS (2141)..(3100)
  3′UTR (3101)..(3195): CDS LacM gene

SEQ ID NO: 15 - protein, 319 aa, Leuconostoc lactis
MAYTNNQLHVIYGDGSLGLQGANFHYLFSYERGGLESLVVNDKEWLYRTPTPMFWRATTDNDHGSGFSVKSAQWYAADKFSTCQDIELTVDDQPVTPLPIAPLNNKYTDHEIATKVSLAYHFVTTTVPSTIVTVTYTVTADGQINIATHYSGQSDLPELPAFGLRFIMPTTATGFDYTGLSGETYPDRLAGATHGQFHVDSLPVTPYLVPQECGMHMQTEQVTVTRSTTQNNADHDNTPFSLTFSQTDAPFAFSCLPYTAAELENATHMEELPLARRTVLSIYGAVRGVGGIDSWGTDVEAPYHILANQDIDFSFNIHF